(19) United States
(12) Patent Application Publication
(10) Pub. No.: US 2020/0129320 A1
(43) Pub. Date: Apr. 30, 2020

(54) SMART GEAR APPARATUS

(71) Applicant: International Business Machines Corporation, Armonk, NY (US)

(72) Inventors: Al Chakra, Apex, NC (US); Yuk L. Chan, Rochester, NY (US); JIAN JUN WANG, XI'AN (CN)

(21) Appl. No.: 16/176,030

(22) Filed: Oct. 31, 2018

(51) Int. Cl. A61F 5/02; G05B 15/02; A41D 1/00; A41D 13/05; A41D 13/06

(52) U.S. Cl. CPC A61F 5/028; G05B 15/02; A41D 1/002; A41D 2400/32; A41D 13/065; A41D 2300/32; A41D 13/0531

(57) ABSTRACT

A smart gear apparatus is provided. The smart gear apparatus includes wearable material, a smart material pad and a control element. The smart material pad is integrated with or at least partially encased in the wearable material and includes smart material having a variable hardness and a control element. The control element is operably coupled to the smart material pad and is configured to adjust the variable hardness of the smart material thereof.

### BACKGROUND

The present invention generally relates to a gear apparatus and, more specifically, to a smart gear apparatus that a person can wear.

People wear gear apparatuses (e.g., waist supports) when they have physical issues and need the type of support certain equipment can provide. It is well understood, however, that one type of equipment or support cannot fit all different types of people and that one type of equipment that can fit a person in recovery time period may not fit that same person in another recovery time period.

### SUMMARY

Embodiments of the present invention are directed to a smart gear apparatus. A non-limiting example of the smart gear apparatus includes wearable material, a smart material pad, and a control element. The smart material pad is integrated with or at least partially encased in the wearable material and includes a smart material having a variable hardness. The control element is operably coupled to the smart material pad and is configured to adjust the variable hardness of the smart material thereof.

Embodiments of the present invention are directed to a smart gear apparatus. A non-limiting example of the smart gear apparatus includes wearable material securable around a body part of an individual, one or more smart material pads and a control element. Each of the one or more smart material pads is integrated with or at least partially encased in one or more support areas of the wearable material. Each of the one or more smart material pads includes a smart material having a hardness which is variable in response to currents of variable intensities being applied thereto. The control element is operably coupled to the one or more smart material pads and is configured to apply the currents of variable intensities to the one or more smart material pads to adjust the hardness of the smart material thereof.

Embodiments of the invention are directed to a method of operation of a smart gear apparatus. A non-limiting example of the smart gear apparatus includes wearable material securable around a body part of an individual, a smart material pad and a control element. The smart material pad is integrated with or at least partially encased in the wearable material and includes a smart material having a hardness which is variable in response to currents of variable intensities being applied thereto. The control element is operably coupled to the smart material pad and is configured to apply the currents of variable intensities to the smart material pad to adjust the hardness of the smart material thereof. A non-limiting example of the method includes controlling the application of the currents by the control element in accordance with one or more of a location of the smart material pad, a predefined schedule, a current activity of the individual and instructions received from a third party.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 is a depiction of a gear apparatus worn by a person as a lower back support in accordance with embodiments of the present invention;

FIG. 2 is a schematic side illustration of a gear apparatus that is wearable by a person as a lower back support in an unrolled condition in accordance with embodiments of the present invention;

FIG. 3 is a schematic top-down illustration of a gear apparatus that is wearable by a person as a lower back support in a rolled condition in accordance with embodiments of the present invention;

FIG. 4 is a cross-sectional view of the gear apparatus of FIGS. 2 and 3 taken along line 4-4 of FIG. 2; and

FIG. 5 is a schematic diagram of a control circuit of a gear apparatus that is wearable by a person as a lower back support in accordance with embodiments of the present invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Turning now to an overview of technologies that are more specifically relevant to aspects of the invention, there are many types of gear apparatuses that are available to consumers for various applications. For example, knee and back braces are available to orthopedic and athletic patients in therapeutic and injury preventing situations. Similar knee and back braces are also available to individuals who regularly lift and move heavy machinery or consumer goods in injury preventing situations. Regardless of the type of brace or the reasons a person might wear them, it is often the case that the brace will not fit all people in all situations. Even for those braces that do fit certain individuals, they may not be usable over long periods of time as the individual's needs change.

Turning now to an overview of the aspects of the invention, one or more embodiments of the invention address the above-described shortcomings of the prior art by providing a smart gear apparatus that includes wearable material, a smart material pad at least partially encased in the wearable material and including smart material having a variable hardness and a control element, which is operably coupled to the smart material pad and which is configured to adjust the variable hardness of the smart material thereof.

The above-described aspects of the invention address the shortcomings of the prior art by providing a gear apparatus in which smart materials thereof are manipulated to change the hardness and softness of smart material pads to thereby allow and support or prevent certain movements of an individual. The manipulation of the smart materials can be based on one or more factors, such as a model analysis, a predefined schedule, a location of the smart material pads, a current activity of the individual, third party instructions, etc.

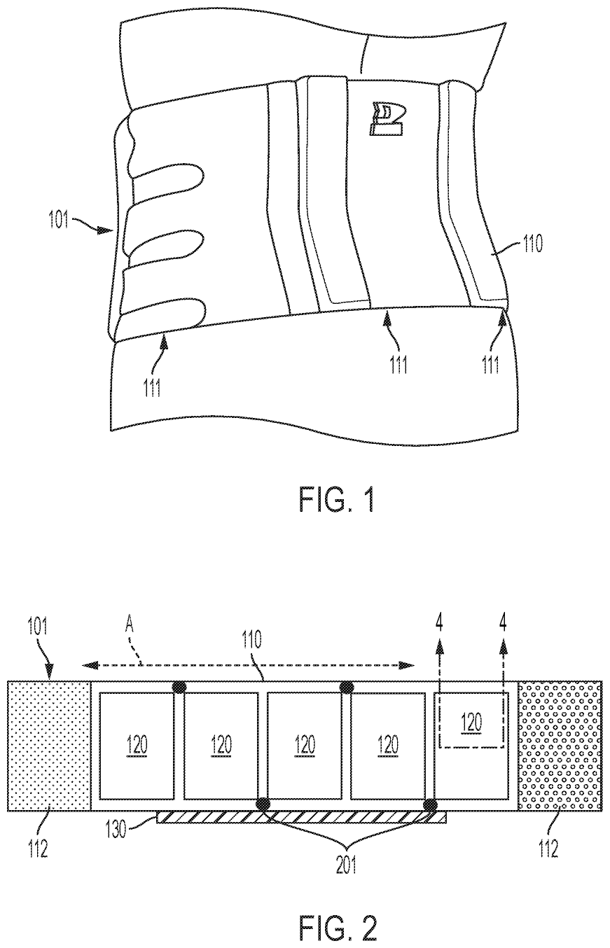

Turning now to a more detailed description of aspects of the present invention, FIGS. 1-3 depict a smart gear apparatus 101. The smart gear apparatus 101 includes wearable material 110, which is securable around a body part, such as an extremity or a lower back (see FIG. 1) of an individual, one or more smart material pads 120 and a control element 130. Each of the one or more smart material pads 120 is integrated with or at least partially encased in one or more support areas 111 of the wearable material 110. In addition, each of the one or more smart material pads 120 includes a smart material having a hardness which is variable in response to currents of variable intensities being applied thereto. Such a smart material is known in the art. The control element 130 is operably coupled to each of the one or more smart material pads 120. The control element 130 is configured to apply the currents of variable intensities to each of the one or more smart material pads 120 in order to adjust the hardness of the smart material thereof.

In accordance with embodiments of the present invention, the wearable material 110 is configured to be securely wrapped around an extremity or a lower back region of the individual. To that end, as shown in FIG. 2, the wearable material 110 can be provided as an elongate strip with opposite ends 112 and 113. Here, the one or more support areas 111 can be arranged along a longitudinal axis A of the wearable material 110 and the opposite ends 112 and 113 can be provided with Velcro™ fasteners or other similar types of fasteners by which the opposite ends 112 and 113 can be secured together. In addition, the wearable material 110 can be formed of one or more of soft material and cushioned material that can be worn by the individual next to his/her skin.

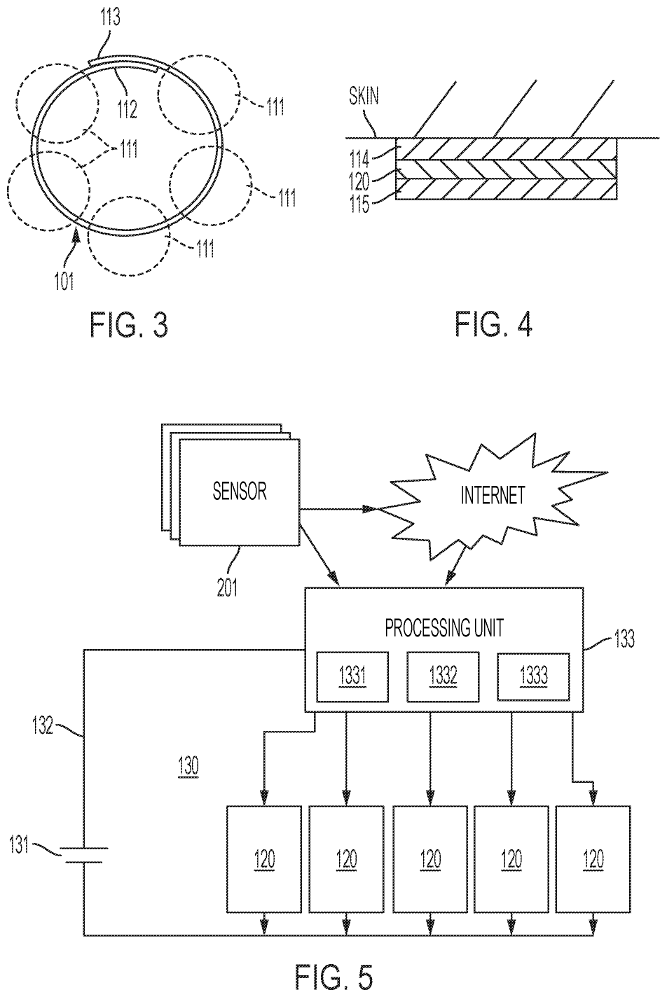

As an additional feature, to the extent that the one or more smart material pads 120 are at least partially encased in the one or more support areas in the wearable material 110, an interior layer 114 (see FIG. 3) of the wearable material prevents skin-smart material pad contact while an exterior layer 115 faces outwardly from the individual.

With such construction, where the smart gear apparatus 101 is provided as a lower back support as shown in FIG. 1, the wearable material 110 can be wrapped securely around the upper waist of the individual and the one or more support areas 111 can be aligned with multiple segments of the individual's waist so that the corresponding one or more smart material pads 120 can sit against the multiple segments of the individual's waist.

As shown in FIG. 2 and with additional reference to FIG. 4, the smart gear apparatus 101 can include one or more sensors 201. The one or more sensors 201 are arrayed throughout the wearable material 110 and are configured to sense various current conditions and environmental characteristics. Each of the one or more sensors 201 can include or be provided as one or more of an accelerometer, a pressure sensor, a temperature sensor, a strain gage or any other sensing element. In addition, each of the one or more sensors 201 can be provided as Internet of Things (IoT) devices. In an exemplary case, the one or more sensors 201 can be configured to cooperatively sense the various current conditions and the environmental characteristics to determine a current activity (e.g., sitting for a long period of time, exercising, doing manual labor, receiving therapy or treatment, etc.) of the individual.

With continued reference to FIG. 4, the control element 130 includes a power source 131, such as a battery or a rechargeable battery, circuitry 132 that is extendable throughout the wearable material 110 and a processing unit 133. The processing unit 133 is operably coupled to the power source 131 and the one or more smart material pads 120 via the circuitry 132. The processing unit 133 includes a processor 1331, a memory unit 1332 and an input/output (I/O) unit 1333 by which the processor 1331 is communicative with the circuitry 132. The memory unit 1332 has executable instructions stored thereon, which are readable and executable by the processor 1331. When the executable instructions are read and executed by the processing 1331, the executable instructions cause the processor 1331 to control an application of the currents of variable intensities from the power source 131 to each of the one or more smart material pads 120.

In accordance with embodiments of the present invention, the processor 1331 can be disposed locally within the control element 130 or disposed remotely from the wearable material 110, the control element 130 and the smart gear apparatus 101 as a whole. In the latter case, the processor 1331 and, in some cases, the memory unit 1332, can be provided within a portable computing device, such as a laptop, a tablet or a smartphone, or within a fixed computing device, such as a desktop computer or a server with the I/O unit 1333 having mobile networking capabilities.

In accordance with various embodiments of the present invention, the processing unit 133 is configured to control the application of the currents to each of the one or more smart material pads 120 in accordance with one or more various factors. The one or more various factors can be defined or informed by the one or more sensors 201 and can include a location of each of the one or more smart material pads 120 relative to the individual's body (e.g., his/her lower back or the side of his/her waist) or relative to a location of the individual in space (e.g., the individual's location in the global positioning system (GPS) or in a particular building), a predefined schedule established for example by the individual's employer, doctor, physical therapist, etc., a current activity of the individual and instructions received from a third party such as the individual's doctor or physical therapist.

For example, if the individual has a lower back problem, his/her doctor may prescribe the use of the smart gear apparatus 101 for support. In such a case, the doctor can control an operation of the smart gear apparatus 101 such that the processing unit 133 controls the application of the currents to each of the one or more smart material pads 120 according to the doctor's plan. For example, at an initial time when the individual's lower back problem is most severe and the doctor wants the individual to have a significant amount of lower back support, the doctor can instruct the processing unit 133 to apply currents with high intensities to the smart material of each of the one or more smart material pads 120 so that the hardness thereof increases. By contrast, at a later time when the individual's lower back problem is less severe and the doctor wants the individual to have more freedom of movement and blood flow, the doctor can instruct the processing unit 133 to apply currents with lower intensities to the smart material of each of the one or more smart material pads 120 so that the hardness thereof decreases.

As another example, if the individual is exercising or repeatedly lifting heavy machinery, the one or more sensors 201 can detect such activity (i.e., from repeated accelerations and pressure readings) and inform the processing unit 133 as to the current activity of the individual. The processing unit 133 can then apply currents with alternating high and low intensities to the smart material of each of the one or more smart material pads 120 so that the hardness thereof increases and decreases during each exercise repetition.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A smart gear apparatus, comprising:
wearable material;
a smart material pad integrated with or at least partially encased in the wearable material and comprising smart material having a variable hardness; and
a control element, which is operably coupled to the smart material pad and which is configured to adjust the variable hardness of the smart material thereof.

2. The smart gear apparatus according to claim 1, wherein the wearable material is configured to be securely wrapped around an extremity or a lower back region of an individual and is one or more of soft and cushioned.

3. The smart gear apparatus according to claim 1, wherein the smart material pad is at least partially encased in the wearable material such that a layer of the wearable material prevents skin-smart material pad contact.

4. The smart gear apparatus according to claim 1, further comprising one or more sensors arrayed throughout the wearable material to sense a current activity of individual using the smart gear apparatus by which the control element is operable.

5. The smart gear apparatus according to claim 1, wherein the control element comprises:
a power source;
circuitry; and
a processing unit operably coupled to the power source and the smart material pad via the circuitry and configured to control an application of currents of variable intensities from the power source to the smart material pad.

6. The smart gear apparatus according to claim 5, wherein the processing unit comprises a processor which is local to or remote from the wearable material.

7. The smart gear apparatus according to claim 5, wherein the processing unit is configured to control the application of the currents to the smart material pad in accordance with a location of the smart material pad.

8. The smart gear apparatus according to claim 5, wherein the processing unit is configured to control the application of the currents to the smart material pad in accordance with a predefined schedule.

9. The smart gear apparatus according to claim 5, wherein the processing unit is configured to receive instructions from a third party and to control the application of the currents to the smart material pad in accordance with the instructions.

10. A smart gear apparatus, comprising:
wearable material securable around a body part of an individual;
one or more smart material pads integrated with or at least partially encased in one or more support areas of the wearable material, each of the one or more smart material pads comprising smart material having a hardness which is variable in response to currents of variable intensities being applied thereto; and
a control element, which is operably coupled to the one or more smart material pads and which is configured to apply the currents of variable intensities to the one or more smart material pads to adjust the hardness of the smart material thereof.

11. The smart gear apparatus according to claim 10, wherein the wearable material is configured to be securely wrapped around an extremity or a lower back region of the individual and is one or more of soft and cushioned.

12. The smart gear apparatus according to claim 10, wherein the one or more smart material pads are at least partially encased in the one or more support areas such that a layer of the wearable material prevents skin-smart material pad contact.

13. The smart gear apparatus according to claim 10, further comprising one or more sensors arrayed throughout the wearable material to sense a current activity of the individual by which the control element is operable.

14. The smart gear apparatus according to claim 10, wherein the control element comprises:
a power source;
circuitry; and
a processing unit operably coupled to the power source and the one or more smart material pads via the circuitry and configured to control an application of the currents of variable intensities from the power source to the one or more smart material pads.

15. The smart gear apparatus according to claim 14, wherein the processing unit comprises a processor which is local to or remote from the wearable material.

16. The smart gear apparatus according to claim 14, wherein the processing unit is configured to control the application of the currents to the one or more smart material pads in accordance with a location of each of the one or more smart material pads.

17. The smart gear apparatus according to claim 14, wherein the processing unit is configured to control the application of the currents to the one or more smart material pads in accordance with a predefined schedule.

18. The smart gear apparatus according to claim 14, wherein the processing unit is configured to control the application of the currents to the one or more smart material pads in accordance with a current activity of the individual.

19. The smart gear apparatus according to claim 14, wherein the processing unit is configured to receive instructions from a third party and to control the application of the currents to the one or more smart material pads in accordance with the instructions.

20. A method of operation of a smart gear apparatus, comprising:
wearable material securable around a body part of an individual;
a smart material pad integrated in or at least partially encased in the wearable material and comprising smart material having a hardness which is variable in response to currents of variable intensities being applied thereto; and
a control element, which is operably coupled to the smart material pad and which is configured to apply the currents of variable intensities to the smart material pad to adjust the hardness of the smart material thereof,
the method comprising:
controlling an application of the currents by the control element in accordance with one or more of a location the smart material pad, a predefined schedule, a current activity of the individual and instructions received from a third party.
